(19) United States
(12) Patent Application Publication
(10) Pub. No.: US 2009/0099532 A1
(43) Pub. Date: Apr. 16, 2009

(54) Assembly for lubricating a portion of a medical device

(76) Inventors: Brian J. Cuevas, Cumming, GA (US); Ali Yahiaoui, Roswell, GA (US); Scott M. Teixeira, Cumming, GA (US); Alison S. Bagwell, Alpharetta, GA (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/974,662

(22) Filed: Oct. 15, 2007

(51) Int. Cl. B65D 85/08; A61M 25/00

(52) U.S. Cl. 604/265; 206/364

(57) ABSTRACT

A medical device assembly for imparting a lubricious and/or antimicrobial liquid onto a medical device is provided. The medical device is surrounded by a pouch and includes a burst package or frangible cells for storage of the liquid prior to delivery onto the surface of the medical device. This helps prevent contamination of the medical device upon removal of the medical device from its pouch, insertion of the device into a body cavity, and implantation of the device into the body cavity.

### BACKGROUND

Medical devices adapted to be inserted into body cavities are commonly used. These devices are often enclosed in permeable or impermeable packages for shipment, storage, and protection from contamination from the outside environment. However, many of these devices may be difficult to insert into body cavities upon removal from the packages.

Many prior art devices have attempted to overcome this problem by prelubricating the device before packaging. However, prelubrication may sometimes cause undesired messiness within the package and may make shipping and handling of the device more complicated. Additionally, pre-lubrication may lose some of its effectiveness because a lubricated item which remains in its package for long periods of time without removal may dry out or because too viscous. Further, many of the prior art devices require that the medical device be precoated with a swellable material and then coated again once it is ready to be removed from the package. This results in an unnecessary added expense.

Many of these prelubricated or unlubricated devices, however, may become contaminated when they are removed from the package and inserted into a body cavity. Although the device is generally provided to the health professional sterile, it is removed and handled in a non-sterile environment. In this regard, a health professional may contaminate the device with his hand or body, microbes from the outside environment may contaminate the medical device, or the medical device may accidentally come into contact with a contaminated surface prior to insertion into a body cavity.

Contamination is of particular concern because when devices such as endotracheal tubes are inserted into the trachea, the likelihood for Ventilator Associated Pneumonia (VAP) increases.

Thus, there remains a need for an economical medical device assembly which provides for lubrication of the medical device for prevention of tissue irritation and bacterial biofilm formation. There is also a need for an economical medical device assembly which reduces the possibility of contamination of the medical device immediately prior to insertion into a body cavity.

### SUMMARY OF INVENTION

The present invention provides for a medical device assembly. The medical device assembly includes a medical device adapted to be coated with a liquid and having a distal and proximal portion. The assembly also includes a pouch which includes a thin flexible region and surrounds the medical device. The pouch may be plastic and/or impermeable. Additionally, the medical device assembly includes a burst package located inside the pouch. The burst package, which may surround the medical device, includes a frangible container that encapsulates liquid and also may include distribution media distribution media such as sponges, nonwovens, cellulosic plies or foam.

Desirably, the medical device may be an endotracheal tube, tracheotomy tube, internal feeding tube, or catheter. The liquid which the medical device is adapted to be coated with may be a polymeric solution such as a phospholipid, collagen, laminin, polyamino acids, carboxymethylcellulose, polyvinylpyrrolidone, chitosan, polyvinylpyroolidone-co-vinyacetate, polyethylene glycol, pluronics, or it may include an active agent such as triclosan, chlorohexidine, charged silver, polyhexamethylene biguanide. The liquid may also include a surfactant or viscosity modifier.

The medical device may be uncoated prior to contact with liquid from the frangible container. Liquid may contact the uncoated medical device directly once released from frangible container or liquid may flow into distribution media where it may be delivered to various portions of the medical device, such as the distal portion.

Another aspect of the invention also provides for a medical device assembly. The medical device assembly includes a medical device adapted to be coated with a liquid and having a distal and proximal portion. The assembly also includes a pouch surround the medical device and including a coherent matrix of frangible cells. Each of the frangible cells is adapted to encapsulate at least one liquid and release the liquid onto the medical device upon palpation of the pouch. It may be desirable, however, that each of the individual cells encapsulates no more than one distinct liquid and that each distinct liquid does not come into contact with another distinct liquid.

Another aspect of the invention provides for a method for coating at least a portion of a medical device prior to insertion into a body cavity. The method includes providing a medical device assembly which includes a medical device adapted to be coated with a liquid and having a distal portion and a proximal portion; providing a pouch surrounding the medical device and including a thin flexible region; and providing a burst package surrounding the medical device. The burst package is located inside the pouch and includes a liquid encapsulating frangible container and distribution media.

The method also includes palpating the distribution media so that the liquid coats at least a portion of the medical device, and removing the coated medical device from the pouch and inserting the coated medical device into a body cavity.

Yet another aspect of the invention provides for a method of providing a system for coating at least a portion of a medical device immediately prior to insertion into a body cavity. The method includes providing a medical device assembly which includes a medical device adapted to be coated with a liquid and having a distal portion and a proximal portion; providing a pouch surrounding the medical device and including a thin flexible region; and providing a burst package surrounding the medical device. The burst package is located inside the pouch and includes a liquid encapsulating frangible container and distribution media.

The method also includes providing a means for palpating the pouch surrounding the medical device so that the liquid encapsulating frangible container breaks and distributes liquid into the distribution media. Further, the method provides a means for palpating the distribution media so that the liquid at least coats at least a portion of the medical device, and a means for removing the coated medical device from the pouch and inserting the coated medical device into a body cavity. The method also includes instructions, with the pouch or separate from the pouch, for palpating the pouch, palpating the distribution media to coat the medical device, and removing the coated medical device from the pouch.

### BRIEF DESCRIPTION OF DRAWINGS

FIG. 1A is a perspective view of a medical device surrounded by a burst package.

FIG. 1B is a cross-sectional view of a burst package surrounding a medical device.

FIG. 2 is a perspective view of a medical device surrounded by a pouch having frangible cells.

### DETAILED DESCRIPTION

The medical device assemblies of the present invention provide for medical device assemblies incorporating medical devices having frangible containers for delivering liquids to various part of a medical device immediately prior to removal of the medical device from its packaging. These liquids provide lubricious coatings and/or antimicrobial protection for the medical devices.

The invention will be described with reference to the following description and figures which illustrate certain embodiments. It will be apparent to those skilled in the art that these embodiments do not represent the full scope of the invention which is broadly applicable in the form of variations and equivalents as may be embraced by the claims appended hereto. Furthermore, features described or illustrated as part of one embodiment may be used with another embodiment to yield still a further embodiment. It is intended that the scope of the claims extend to all such variations and embodiments.

In the interests of brevity and conciseness, any ranges of values set forth in this specification contemplate all values within the range and are to be construed as support for claims reciting any sub-ranges having endpoints which are whole number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

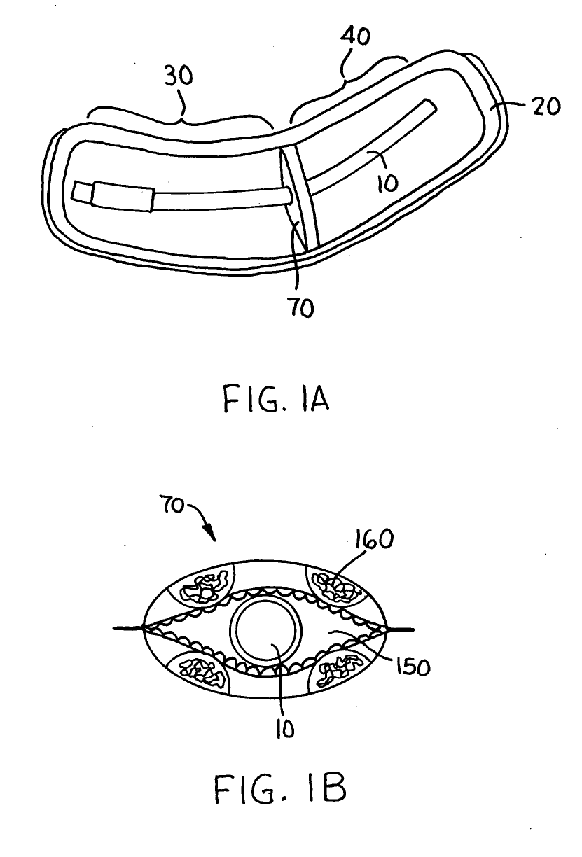

Referring to FIGS. 1A and 1B, a medical device assembly is provided. The medical device assembly includes a medical device 10 having distal 30 and proximal 40 portions. The medical device may be any medical device that may be adapted for insertion into a body cavity. These devices include, but are not limited to, endotracheal tube, tracheotomy tube, internal feeding tube, or catheters. Medical devices for use with the present invention may also include devices which are not designed for insertion into a body cavity.

Returning to FIG. 1A, a pouch 20 surrounds the medical device. The pouch may include any material capable of providing some degree of protection against the outside atmosphere during shipment or storage of the medical device. These materials include, but are not limited to, plastic. The pouch 20 may be permeable or impermeable, though desirably the pouch may be permeable so it may be sterilized. The pouch may be any size or shape which enables the medical device to not be directly exposed to the outside atmosphere, such as, for example, square or rectangular. Additionally, a second pouch or cover is not needed to surround the medical device in order to maintain the position of the medical device within the pouch. In this regard, an optional plug or connector may be integrated with the packaging which is adapted to connect with the distal end of the medical device and hold it in place within the pouch.

The medical device includes a burst package 70 surrounding at least a portion of it. The burst package will act as a divider between the distal 30 and proximal 40 sections of the device and will be in communication with the pouch and the medical device. The burst package includes a frangible container 160 and distribution media 150. The frangible container contains liquid which is adapted to be delivered to portion of the medical device, desirably the distal end, upon palpation of the frangible container. The frangible container may be constructed of the same material as the pouch, i.e. plastic. However, any material suitable for containing liquid during storage and shipping of the medical device assembly may also be used.

Upon palpation of the frangible container, liquid may be delivered directly to the medical device or liquid may flow into the distribution media for delivery to a portion of the medical device, i.e, the distal portion. The distribution may be any device suitable for delivering fluids to a portion of the medical device, i.e. a sponge, foam, nonwoven, ply or plies of cellulose, brush, or flexible ring. In this regard, liquid delivered from the frangible container, by applying pressure to the frangible container through hand, fingers, or other effective means, may flow into the distribution media. The distribution media may then act as a squeegee. i.e. in the case of a sponge, for transporting liquids to a portion of the medical device. Alternatively, in particular situations, the distribution media may allow for flow of the liquid from the frangible container onto a portion of the medical device without the use of a squeegee mechanism or may coat at least a portion of the medical device upon removal of the medical device from the pouch. In this regard, upon removal of the medical device from the pouch, a health professional, will grab the proximal end of the device to remove the device from the pouch and the distal portion will be coated further as it is slidably removed from the pouch. Although it is desirable to coat the distal portion of the medical device because it is generally inserted into the body cavity, any portion of the medical device may be coated.

It is contemplated that the liquid distributed to a portion of the medical device may act as a lubricious coating for the device when it is inserted into a body cavity. This provides for easier insertion of the medical device into the body cavity and decreases the chances that insertion will result in irritation of the tissues of the body cavity and an increase in the likelihood of entrance of pathogens into the body through the delicate tissues of the body cavity. In this regard, the coatings are designed to provide short term protection, i.e. the time required implantation of the device. This short term protection may range from about 1 minute up to about 2 weeks, desirably between 1 minute and 1 hour. It is also contemplated that the short term protection may last longer than 2 weeks. Desirably, after implantation, the coating or coatings will be flushed away by the natural secretions of the body.

In addition to use as a lubricious coating, the coating may be used to impart antibacterial and anti-microbial properties to the medical device. In this regard, certain liquids may prevent bacteria from forming on the surface of the medical device prior to insertion into the body cavity and/or kill microbes and bacteria that colonize on the medical device prior to insertion in the body cavity. Further, even after insertion into the body cavity, some liquids may deactivate bacteria and microbes and decrease incidences of VAP. Desirably, when the lubricious coating has antimicrobial or antibacterial properties, it will also offer short term protection against microbes from the environment. As previously mentioned, short term protection may range from about 1 minute up to about 2 weeks, desirably between 1 minute and 1 hour. It is also contemplated that the short term protection may last longer than 2 weeks.

Importantly, whether a liquid has anti-microbial properties or not, each of these liquids does not require the medical device to be coated prior to coating the medical device with a specific formulation or formulations, unlike some prior art medical devices which require the application of a swellable coating to the device prior to the application of an additional liquid. This decreases the economic costs of providing lubrication and/or antibacterial protection for the medical device. Additionally, because in some situations the pouch may have some degree of permeability, microbes may enter the pouch through the atmosphere during shipment or storage. If a coating is adhered to the surface of the medical device during this time, microbes may attach to the surface and colonize within the coating. By utilizing an uncoated medical device which is only coated immediately prior to implantation, the possibility of bacterial biofilm formation during storage and shipment is significantly reduced.

Liquids suitable for use with the present invention include polymeric solutions. Non-limiting examples of polymeric solutions include phospholipid, acrylic acid polymer, collagen, laminin, polyamino acids, carboxymethylcellulose, polyvinylpyrrolidone, chitosan, polyvinylpyroolidone-co-vinyacetate, polyethylene glycol, pluronics, or combinations thereof.

Additionally, various other liquids may be used including silicon based water soluble lubricants such as alkylene oxide modified silicone glycol. Further, liquids such as lecithin, water, water soluble gums, saline solutions, oil, colloidal suspensions, and emulsions including oil-water-emulsions may be used. However, any liquid which is biocompatible and which provides a lubricious coating to the medical device for insertion into a body cavity is appropriate.

Regardless, of the type of liquid used, various additives may be used. For example, active agents, with optional surfactants, may be added to the liquid. The active agents kill or deactivate microbes and bacteria upon contact or within a short period of time. These active agents include, but are not limited to, triclosan, chlorohexidine, charged silver, polyhexamethylene biguanide, and combinations thereof. Surfactants may be used with active agents to aid in the profusion of the active agent into the microbial or bacterial cell wall, thus aiding the active agent's ability to kill or deactivate the microbe or bacteria. Non-limiting examples of surfactants include copolymers of polysiloxane and polyoxyethylene.

Viscosity modifiers, as known in the art, may also be used with liquids. Viscosity modifiers may function to reduce surface friction of and form a water multilayer that may reduce the affinity for bacteria to attach to the medical device.

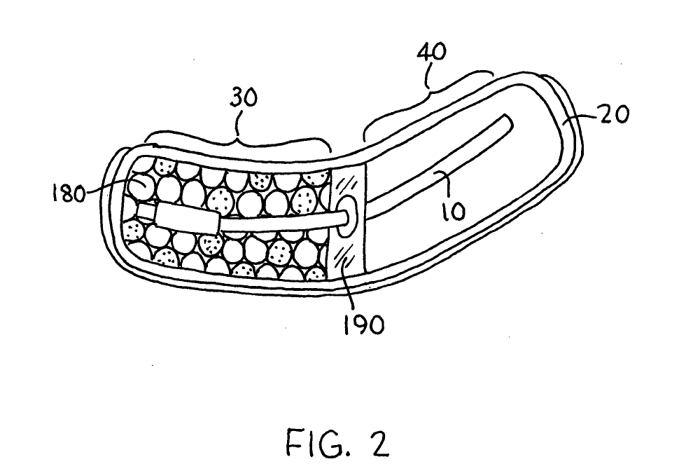

Turning, to FIG. 2, a medical device assembly having a pouch 20 surrounding the medical device 10 is provided. The medical device 10 includes a barrier 190 surrounding it. The barrier separates the distal portion 30 of the medical device from the proximal portion of the medical device 40.

The pouch 20 surrounding the medical device includes a coherent matrix of frangible cells surrounding it (similar to a bubble wrap assembly). These frangible cells contain liquid which may be distributed onto the medical device upon palpation of a frangible cell or frangible cells. Desirably, the frangible cells are located on the area of the pouch corresponding to the distal portion of the medical device in order to transfer liquid onto the medical device prior to removal of the device from the pouch by way of handling of the proximal end of the device by a health professional or other individual. The frangible cells, however, may be located on any portion of the pouch.

Each individual cell will enclose one liquid, a combination of liquids, or no liquid at all within the cell. Thus, discrete portions of the medical device may be coated with different liquids, combination of liquids, or no liquids at all upon palpation of an individual cell or combinations of cells.

This arrangement of cells could prove particularly important where discrete locations of cells correspond to anatomical locations within a body cavity. For example, it is contemplated that when an endotracheal tube is inserted into the trachea, individual sections of the trachea may be susceptible to various and different infections. Thus, an individual cell or group of cells may contain within them liquids that are most effective for treating the anatomical location of the tracheal cavity corresponding to the location of the frangible cell.

Additionally, by having multiple frangible cells containing liquid, novel combinations of liquids may be able to be used together for lubricious coatings and/or antimicrobial or antibacterial effect. For example, in situations where two or more liquids cannot be stored together for long periods of time, the liquids could be placed in adjacent frangible cells, palpated immediately prior to use, and inserted into the body cavity before the combination of liquids loses its effectiveness or becomes volatile. Further, individual liquids may be found in adjacent cells and not be combined or mixed upon palpation of the cells.

In addition to the medical device assemblies and methods provided above, the present invention encompasses a method of providing a system for coating at least a portion of a medical device immediately prior to insertion into a body cavity. Generally speaking the system includes providing a medical device assembly; providing a means for palpating a pouch surrounding the medical device so that liquid encapsulating frangible containers break and distribute liquid into the distribution media; providing a means for palpating the distribution media so that the liquid at least coats at least a portion of the medical device; and providing a means for removing the coated medical device from the pouch and inserting the coated medical device into a body cavity.

The medical device assembly includes a medical device adapted to be coated with a liquid and having a distal portion and a proximal portion; a pouch at least partially comprising a thin flexible region, the pouch surrounding the medical device; and a burst package located inside the pouch, the burst package surrounding the medical device and including a liquid encapsulating frangible container and distribution media. The pouch may be any shape or size that encompasses the medical device.

The system may also include instructions for palpating the pouch, palpating the distribution media to coat the medical device, and removing the coated medical device from the pouch. In this regard, palpation of the pouch may be accomplished with a hand, finger, or any other part of the body capable of providing enough force to rupture the frangible container of the burst package or may be accomplished through other physical means. Additionally, the medical device may be removed from the pouch with a hand, finger, other body part, or other physical means. The instructions may be included within the pouch or outside the pouch.

We claim:

1. A medical device assembly comprising:
A medical device adapted to be coated with a liquid and having a distal portion and a proximal portion;
a pouch, at least a portion of the pouch comprising a thin flexible region, wherein the pouch surrounds the medical device; and
a burst package located inside the pouch and including a frangible container and distribution media.

2. The medical device assembly of claim 1, wherein the medical device is an endotracheal tube, tracheotomy tube, internal feeding tube, or catheter.

3. The medical device assembly of claim 1, wherein the pouch is gas impermeable.

4. The medical device assembly of claim 1, wherein the pouch comprises plastic.

5. The medical device assembly of claim 1, wherein the distribution media comprises a sponge, foam, a nonwoven, or cellulose.

6. The medical device assembly of claim 1, wherein the frangible container encapsulates a liquid and the liquid is adapted to coat at least a portion of the medical device.

7. The medical device assembly of claim 6, wherein the liquid comprises a polymeric solution.

8. The medical device assembly of claim 7, wherein the polymeric solution is a phospholipid, collagen, laminin, polyamino acids, carboxymethylcellulose, polyvinylpyrrolidone, chitosan, polyvinylpyroolidone-co-vinyacetate, polyethylene glycol, pluronics, or combinations thereof.

9. The medical device assembly of claim 6, wherein the liquid comprises an active agent.

10. The medical device assembly of claim 9, wherein the active agent is triclosan, chlorohexidine, charged silver, polyhexamethylene biguanide, and combinations thereof.

11. The medical device assembly of claim 6, wherein the liquid comprises a surfactant.

12. The medical device assembly of claim 6, wherein the liquid comprises a viscosity modifier.

13. The medical device assembly of claim 6, wherein the liquid encapsulated by the frangible container is adapted to coat at least a portion of the medical device upon palpation of the pouch to break the frangible container.

14. The medical device assembly of claim 13, wherein liquid contacts the distribution media located adjacent the medical device on the proximal portion of the medical device, prior to coating at least a portion of the medical device.

15. The medical device assembly of claim 14, wherein the distribution media is adapted to deliver the liquid to the distal portion of the medical device.

16. The medical device assembly of claim 1, wherein the medical device is uncoated.

17. The medical device assembly of claim 1, wherein the burst package surrounds at least a portion of the medical device.

18. A medical device assembly comprising:
A medical device having a distal portion and a proximal portion;
a pouch surrounding the medical device, at least a portion of the pouch comprising a coherent matrix of frangible cells; and
at least one liquid encapsulated by at least one individual frangible cell, the cell or cells being adapted to release liquid onto the medical device upon palpation of the pouch.

19. The medical device assembly of claim 18 wherein the medical device is an endotracheal tube, tracheotomy tube, internal feeding tube, or catheter.

20. The medical device assembly of claim 18, wherein the pouch is gas impermeable.

21. The medical device assembly of claim 18, wherein the pouch comprises plastic.

22. The medical device assembly of claim 18, wherein the at least one liquid comprises at least one polymeric solution.

23. The medical device assembly of claim 22, wherein the at least one polymeric solution is a phospholipid, collagen, laminin, polyamino acids, carboxymethylcellulose, polyvinylpyrrolidone, chitosan, polyvinylpyroolidone-co-vinyacetate, polyethylene glycol, pluronics, and combinations thereof.

24. The medical device assembly of claim 18, wherein the at least one liquid comprises at least one active agent.

25. The medical device assembly of claim 24, wherein the at least one active agent is triclosan, chlorohexidine, charged silver, polyhexamethylene biguanide, and combinations thereof.

26. The medical device assembly of claim 18, wherein the at least one liquid comprises at least one surfactant.

27. The medical device assembly of claim 18, wherein the at least one liquid comprises at least one viscosity modifier.

28. The medical device assembly of claim 18, wherein each individual cell encapsulates a distinct liquid.

29. The medical device assembly of claim 28, wherein upon palpation of the pouch, each distinct liquid does not come into contact with another distinct liquid.

30. A method for coating at least a portion of a medical device immediately prior to insertion into a body cavity, the method comprising:
Providing a medical device assembly comprising;
A medical device adapted to be coated with a liquid and having a distal portion and a proximal portion;
a pouch at least partially comprising a thin flexible region, the pouch surrounding the medical device; and
a burst package located inside the pouch, the burst package surrounding at least a portion of the medical device and including a liquid encapsulating frangible container and distribution media;
Palpating the pouch surrounding the medical device so that the liquid encapsulating frangible container breaks and distributes liquid into the distribution media;
Palpating the distribution media so that the liquid coats at least a portion of the medical device; and
Removing the coated medical device from the pouch and inserting the coated medical device into a body cavity.

31. A method of providing a system for coating at least a portion of a medical device immediately prior to insertion into a body cavity, the system comprising:
Providing a medical device assembly comprising;
A medical device adapted to be coated with a liquid and having a distal portion and a proximal portion;
a pouch at least partially comprising a thin flexible region, the pouch surrounding the medical device; and
a burst package located inside the pouch, the burst package surrounding the medical device and including a liquid encapsulating frangible container and distribution media;
Providing a means for palpating the pouch surrounding the medical device so that the liquid encapsulating frangible container breaks and distributes liquid into the distribution media;
Providing a means for palpating the distribution media so-that the liquid at least coats at least a portion of the medical device; and
Providing a means for removing the coated medical device from the pouch and inserting the coated medical device into a body cavity.

32. The method of claim 31, wherein the medical device assembly further comprises instructions for palpating the pouch, palpating the distribution media to coat the medical device, and removing the coated medical device from the pouch.

33. The method of claim 32, wherein instructions for palpating the pouch, palpating the distribution media to coat the medical device, and removing the coated medical device from the pouch are provided separately from the medical device assembly.
